(19) United States
(12) Patent Application Publication
(10) Pub. No.: US 2026/0091074 A1
(43) Pub. Date: Apr. 2, 2026

(54) Composition for regulation of autonomic nervous system

(71) Applicant: N.V. Nutricia, Zoetermeer (NL)

(72) Inventors: Jinhui Hu, Shanghai (CN); Jiangyi Mao, Shanghai (CN); Yi Wang, Shanghai (CN); Yi Jin, Shanghai (CN); Mengjin Liu, Shanghai (CN); Gregg Ward, Shanghai (CN)

(73) Assignee: N.V. Nutricia, Zoetermeer (NL)

(21) Appl. No.: 19/411,410

(22) Filed: Dec. 8, 2025

Related U.S. Application Data
(63) Continuation of application No. PCT/CN2024/097895, filed on Jun. 7, 2024.

(30) Foreign Application Priority Data
Jun. 9, 2023 (WO) PCT/CN2023/099462

(51) Int. Cl. A61K 36/725; A61K 31/197; A61K 31/202; A61K 31/355; A61K 31/375; A61K 31/4415; A61K 31/519; A61K 31/714; A61P 11/00; A61P 25/00; A61P 25/22; A61P 25/24; A61P 25/28; A61P 43/00

(52) U.S. Cl. CPC A61K 36/725; A61K 31/197; A61K 31/202; A61K 31/355; A61K 31/375; A61K 31/4415; A61K 31/519; A61K 31/714; A61P 11/00; A61P 25/00; A61P 25/22; A61P 25/24; A61P 25/28; A61P 43/00

(57) ABSTRACT

The invention relates to a composition for use in regulating the autonomic nervous system.

### TECHNICAL FIELD OF THE INVENTION

The invention relates to a composition for use in regulating the autonomic nervous system.

### BACKGROUND OF THE INVENTION

The autonomic nervous system of humans is part of the peripheral nervous system and contributes to the maintenance of homeostasis by regulating a range of involuntary physiological processes. The autonomic nervous system regulates functions such as cardiac function, involuntary respiration, hormone secretion, body temperature, blood flow and pressure, digestion and metabolism. The autonomic nervous system plays a role in most physiological systems, for example cardiovascular system or gastrointestinal system. Therefore, regulation of autonomic nervous system is relevant in a number of conditions. In particular, regulation of autonomic nervous system plays a role in the treatment of respiration issues, and in thermoregulation.

Thermoregulation is the mechanism by which the internal body temperature is maintained constant at a physiological temperature. In humans internal body temperature depends on where it is measured and varies during the day. Nevertheless, physiological internal body temperature for humans is considered to be around 37° C. However, in certain conditions humans may experience an increase or decrease of body temperature which yields symptoms such as feeling too hot or too cold, hot flashes, night sweats, shivering. Hormonal changes are a major factor in body temperature regulation and symptoms of feeling too hot or too cold. For example, human subjects suffering from hormonal imbalances (such as perimenopausal/menopausal women) and human subjects undergoing hormonal treatments may suffer from an imbalance in thermoregulation. These individuals would greatly benefit from a treatment for regulating the autonomic nervous system, in particular from improved thermoregulation.

US 2012/015882 discloses an orally ingestible composition for regulating an autonomic nervous activity. The composition comprises as active ingredient a peptide Tyr-Pro or a salt thereof.

EP 2143719 discloses a pharmaceutical composition, a food or a beverage containing 2,3-dihydro-3,5-dihydroxy-6-methyl-4H-pyran-4-one, which have an autonomic nervous control effect.

The prior art focuses on pharmaceutical synthetic ingredients, which are generally expensive to produce. There remains a need in the art for a composition comprising nutritional ingredients that subjects can consume together with their normal diet, and that is capable of regulating the autonomic nervous system.

### SUMMARY OF THE INVENTION

The inventors have surprisingly found that a composition comprising a combination of γ-aminobutyric acid (GABA) and jujube improves autonomic nervous system regulation in a human subject. More specifically, the combination of GABA and jujube improved thermoregulation (bidirectional) and improved breathing in the human subject. By the improved breathing the human subject also experiences less snoring.

Through a clinic study and survey the inventors have surprisingly found that the composition of the invention has presented significant benefits in regulating the autonomic nervous system. Such benefits are believed to bring positive effects in treating and/or preventing one or more of hyperlipidaemia, hypertension, diabetes, obesity, overweight, stress, anxiety, depression, and memory loss.

γ-aminobutyric acid is an inhibitory neurotransmitter, that reduces neuronal excitability. Without wishing to be bound by a theory, the inventors believe that jujube increases the inhibitory effect of GABA.

Hence, the invention pertains to the use of a combination of
(i) γ-aminobutyric acid,
(ii) jujube,
   in the manufacture of a composition for regulating the autonomic nervous system in a human subject.

The invention further relates to the use of a combination of
(i) γ-aminobutyric acid,
(ii) jujube,
   in the manufacture of a composition for treating and/or preventing one or more of stress, anxiety, depression, and memory loss in a human subject.

Lastly, the invention pertains to a scoring method for regulating the autonomic nervous system in a human subject, comprising administering daily to said human subject a composition comprising
(i) γ-aminobutyric acid,
(ii) jujube, and
   wherein the method further comprises applying a wellbeing scoring system with the following steps:
a. obtaining user information from the human subject;
b. analyzing the obtained user information to obtain a wellbeing analysis;
c. based on the outcome of the wellbeing analysis:
   providing a wellbeing score to the human subject; and/or
   providing one or more suggestions for behavioral change to the human subject;
      wherein steps a. to c. are repeated at least daily.

### DETAILED DESCRIPTION OF THE INVENTION

A first aspect of the invention thus concerns the use of a combination of
(i) γ-aminobutyric acid,
(ii) jujube,
   in the manufacture of a composition for regulating the autonomic nervous system in a human subject.

For some jurisdictions, the invention may also be worded as a composition comprising
(i) γ-aminobutyric acid,
(ii) jujube,
   for use in regulating the autonomic nervous system in a human subject.

For some other jurisdictions, the invention may also be worded as a method for regulating the autonomic nervous system in a human subject, said method comprising the administration to said human subject of a composition comprising
(i) γ-aminobutyric acid,
(ii) jujube.

For some jurisdictions, ‘regulating the autonomic nervous system’ may be considered non-therapeutic. By definition, the words ‘non-therapeutic’ exclude any therapeutic effect. Thus, for some jurisdictions, the invention may be worded as a non-therapeutic method of regulating the autonomic nervous system in a human subject, said method comprising the administration to said human subject of a composition comprising
(i) γ-aminobutyric acid,
(ii) jujube.

A second aspect of the invention concerns the use of a combination of
(i) γ-aminobutyric acid,
(ii) jujube,
   in the manufacture of a composition for treating and/or preventing one or more of hyperlipidaemia, hypertension, diabetes, obesity, overweight, stress, anxiety, depression, and memory loss in a human subject, preferably treating and/or preventing one or more of stress, anxiety, depression and memory loss in a human subject.

For some jurisdictions, the second aspect of the invention may also be worded as a composition comprising
(i) γ-aminobutyric acid,
(ii) jujube,
   for use in treating and/or preventing one or more of hyperlipidaemia, hypertension, diabetes, obesity, overweight, stress, anxiety, depression, and memory loss in a human subject, preferably treating and/or preventing one or more of stress, anxiety, depression and memory loss in a human subject.

For some other jurisdictions, the second aspect of the invention may also be worded as a (non-therapeutic) method for treating and/or preventing one or more of hyperlipidaemia, hypertension, diabetes, obesity, overweight, stress, anxiety, depression, and memory loss in a human subject, preferably treating and/or preventing one or more of stress, anxiety, depression and memory loss in a human subject,
said method comprising the administration to said human subject of a composition comprising
   (i) γ-aminobutyric acid,
   (ii) jujube.

In the context of the invention, the following definitions are provided.

The term ‘jujube’ as used herein refers to the plant Ziziphus jujuba, which is also known as Chinese date, spine date, spina date or red date. The term ‘jujube’ as used herein encompasses any part of the plant, fruit, leaves, seeds or extracts thereof, unless indicated otherwise. Jujube seed extract is also known as suanzoaren.

The term ‘thermoregulation’ as used herein refers to the ability of a living being of maintaining its internal temperature constant around physiological temperature. In humans body temperature depends on where it is measured and varies during the day. Physiological internal body temperature for humans is considered to be 37° C.±1° C. Bidirectional thermoregulation as used herein refers to the ability to maintain internal temperature from either direction of above 37° C. (too hot) or below (too cool).

The term ‘lipid’ as used herein comprises one or more selected from the group consisting of triglycerides, free fatty acids, monoglycerides and diglycerides and polar lipids. The terms ‘lipid’, ‘fat’ and ‘oil’ are used interchangeably herein and have equal meaning.

Preferably, the human subject is at least 30 years of age, more preferably at least 40 years of age, most preferably at least 50 years of age.

In one preferred embodiment, regulating the autonomic nervous system in a human subject is improving thermoregulation. ‘Improving thermoregulation’ as used herein, preferably encompasses lowering, maintaining and increasing body temperature around physiological body temperature. Improved thermoregulation preferably encompasses the treatment of one or more symptoms selected from feeling too hot, feeling too cold, dry mouth, hot rash, hyperhidrosis, goose bumps, night sweats, and hot flashes. Preferably, the human subject is one or more of a human subject suffering of hormonal imbalance, a human subject undergoing hormonal treatments, a human subject who is underweight, a human subject who is overweight, or an aging human subject. Preferably, the human subject suffering of hormonal imbalance is selected from a woman in perimenopausal period or a woman in menopausal period. Thermoregulation is important for personal comfort as well as being beneficial for a number of other performance functions.

In a second preferred embodiment, regulating the autonomic nervous system in a human subject is improving breathing and/or treating snoring. ‘Improving breathing’ preferably encompasses one of more of having less difficulty with breathing, being less out of breath, reducing chest tightness and treating sleep apnea. More preferably, ‘Improving breathing’ encompasses one of more of having less difficulty with breathing, being less out of breath and reducing chest tightness.

Preferably the composition is orally administered to the human subject.

The inventors surprisingly found that administration of the composition within one hour before going to bed is preferred to bring the positive technical effect described herein on the regulation of autonomic immune system. Preferably the composition is administered to the human subject at least 0.5 hours before going to bed, more preferably between 0.5 and 1.5 hours before going to bed and most preferably between 0.75-1.25 hours before going to bed.

Preferably the composition is administered to the human subject once per day.

Preferably the composition is administered to the human subject for a period of at least 7 days, more preferably for a period of at least 14 days.

### γ-Aminobutyric Acid

γ-aminobutyric acid (GABA) as used herein is also known as 4-aminobutanoic acid, and has CAS number 56-12-2.

Preferably the composition provides a daily dosage of γ-aminobutyric acid of 15-150 mg per day, more preferably 30-130 mg per day, most preferably 45-120 mg per day.

Preferably the amount of γ-aminobutyric acid in the composition is 100-1000 mg per 100 g of dry weight of the composition, more preferably 150-800 mg per 100 g of dry weight of the composition, most preferably 200-600 mg per 100 g of dry weight of the composition.

### Jujube

By adding plant-based and traditional nutrition jujube, the composition achieved not only physical and psychological benefits, but also economic and nutritional balance. Therefore, the composition is typically more acceptable and user-friendly to Asian and Chinese consumers.

Preferably, jujube encompasses any part of the plant Ziziphus jujuba. More preferably, jujube is provided as jujube fruit, jujube leaves, jujube seed, or combinations thereof. Even more preferably, jujube is provided as jujube seed. Most preferably, jujube is provided as jujube seed powder.

Preferably the weight ratio of γ-aminobutyric acid (mg) to jujube (mg) is in the range of 2:1-1:10, preferably 1:1-1:8, more preferably 1:2-1:6.

Preferably the composition provides a daily dosage of jujube of 100-1000 mg per day, more preferably 150-800 mg per day, most preferably 200-700 mg per day.

Preferably the amount of jujube in the composition is 0.1-6 g per 100 g of dry weight of the composition, more preferably 0.3-5 g per 100 g dry weight of the composition, most preferably 0.5-4 g per 100 g dry weight of the composition.

### Protein

Preferably the composition further comprises protein.

Preferably the weight ratio of γ-aminobutyric acid (mg) to protein (g) is in the range of 30:1-5:1, preferably 25:1-10:1.

Preferably the composition provides a daily dosage of protein of 3-9 g per day, more preferably 4.5-7.5 g per day, most preferably 5.5-6.5 g per day.

Preferably the amount of protein in the composition is 5-45 g per 100 g of dry weight of the composition, more preferably 15-35 g per 100 g of dry weight of the composition, most preferably 20-30 g per 100 g of dry weight of the composition.

Preferably the protein is dairy protein. More preferably the dairy protein is whey protein, casein or a combination thereof. Dairy protein is preferably provided in any suitable form, for example dairy protein may be provided by acid whey, sweet whey, whey protein concentrate, whey protein isolate, caseinate, milk powder, skim milk powder, whole milk powder, or combinations thereof. Preferably, dairy protein is provided by whole milk powder.

### Lipid

Preferably the composition further comprises lipid.

Preferably the composition provides a daily dosage of lipid of 3-10 g per day, more preferably 5-8 g per day, most preferably 6-7 g per day.

Preferably the amount of lipid in the composition is 5-45 g per 100 g of dry weight of the composition, more preferably 15-35 g per 100 g of dry weight of the composition, most preferably 20-30 g per 100 g of dry weight of the composition.

The lipid preferably comprises polyunsaturated fatty acids (PUFA). Preferably, the PUFA comprise omega-3 and/or omega-6 PUFA. More preferably the PUFA comprise one or more of docosahexaenoic acid (DHA), eicosapentaenoic acid (EPA), and docosapentaenoic acid (DPA). Even more preferably, the PUFA comprise DHA and/or EPA, and most preferably the PUFA comprise both DHA and EPA.

Preferably the composition provides a daily dosage of DHA of 1-50 mg per day, more preferably 2-35 mg per day, most preferably 5-15 mg per day.

Preferably the amount of DHA in the composition is 4-200 mg per 100 g of dry weight of the composition, more preferably 8-140 mg per 100 g dry weight of the composition, most preferably 20-60 mg per 100 g dry weight of the composition.

Preferably the composition provides a daily dosage of EPA of 1-50 mg per day, more preferably 3-35 mg per day, most preferably 5-20 mg per day.

Preferably the amount of EPA in the composition is 4-200 mg per 100 g of dry weight of the composition, more preferably 8-140 mg per 100 g of dry weight of the composition, most preferably 20-80 mg per 100 g dry weight of the composition.

Preferably the weight ratio of DHA (mg) to EPA (mg) is in the range of 5:1-1:4, preferably 3:1-1:3, more preferably in the range of 2:1-1:2, most preferably 1:1-1:1.5.

Preferably the lipid comprises microbial oil, fish oil, milk fat, or combinations thereof.

It is preferred that the lipid comprises fish oil and milk fat. Preferably the weight ratio of fish oil (g) to milk fat (g) is in the range of 1:20-1:80, more preferably 1:30-1:70, most preferably 1:40-1:60.

Preferably the composition comprises a fish oil comprising 5-30 wt. % EPA, by weight of fatty acids. More preferably, the composition comprises a fish oil comprising 10-25 wt. % EPA, by weight of fatty acids.

Preferably the amount of fish oil in the composition is 0.1-4 g per 100 g of dry weight of the composition, more preferably 0.2-3 g per 100 g of dry weight of the composition, most preferably 0.2-2 g per 100 g dry weight of the composition.

The milk fat is preferably provided as anhydrous milk fat, butter oil, butter fat, butter, whole milk powder or combinations thereof. More preferably, the milk fat is provided by whole milk powder.

Preferably the amount of milk fat in the composition is 5-45 g per 100 g of dry weight of the composition, more preferably 15-35 g per 100 g of dry weight of the composition, most preferably 20-30 g per 100 g dry weight of the composition.

### Vitamins and Minerals

Preferably the composition further comprises vitamins.

Preferably the vitamins are selected from folate, vitamin B6, vitamin B12, vitamin C, vitamin E, or a combination thereof.

Preferably the composition provides a daily dosage of folate of 10-300 μg per day, more preferably 50-250 μg per day, most preferably 100-200 μg per day. Preferably the amount of folate in the composition is 100-1200 μg per 100 g of dry weight of the composition, more preferably 200-1000 μg per 100 g of dry weight of the composition, most preferably 400-700 μg per 100 g of dry weight of the composition.

Preferably the composition provides a daily dosage of vitamin B6 of 100-600 μg per day, more preferably 200-500 μg per day, most preferably 300-400 μg per day. Preferably the amount of vitamin B6 in the composition is 0.1-20 mg per 100 g of dry weight of the composition, more preferably 0.2-15 mg per 100 g of dry weight of the composition, most preferably 0.4-10 mg per 100 g of dry weight of the composition.

Preferably the composition provides a daily dosage of vitamin B12 of 0.1-20 μg per day, more preferably 0.2-10 μg per day, most preferably 0.4-5 μg per day. Preferably the amount of vitamin B12 in the composition is 0.1-40 μg per 100 g of dry weight of the composition, more preferably 0.2-20 μg per 100 g of dry weight of the composition, most preferably 0.4-10 μg per 100 g of dry weight of the composition.

Preferably the composition provides a daily dosage of vitamin C of 1-100 mg per day, more preferably 5-75 mg per day, most preferably 10-50 mg per day. Preferably the amount of vitamin C in the composition is 10-300 mg per 100 g of dry weight of the composition, more preferably 30-250 mg per 100 mg of dry weight of the composition, most preferably 50-200 mg per 100 g of dry weight of the composition.

Preferably the composition provides a daily dosage of vitamin E (expressed in α-tocopherol equivalent [α-TE]) of 0.1-20 mg α-TE per day, more preferably 0.5-15 mg α-TE per day, most preferably 1-10 α-TE mg per day. Preferably the amount of vitamin E is 1-200 mg α-TE per 100 g of dry weight of the composition, more preferably 2-100 mg α-TE per 100 g of dry weight of the composition, most preferably 4-50 mg per 100 g α-TE of dry weight of the composition.

Preferably the composition further comprises calcium. Preferably the composition provides a daily dosage of calcium of 50-500 mg per day, more preferably 100-400 mg per day, most preferably 150-300 mg per day. Preferably the amount of calcium in the composition is 400-1200 mg per 100 g of dry weight of the composition, more preferably 600-1000 mg per 100 g of dry weight of the composition, most preferably 700-900 mg per 100 g of dry weight of the composition.

### Composition

The composition is preferably in the form of a supplement, a concentrated liquid, a ready-to-drink composition or a powder. More preferably the composition is a powder. Preferably, the powder is to be reconstituted with an aqueous liquid to prepare a liquid formula, this liquid formula is subsequently orally administered to the human subject. Preferably, the aqueous liquid is water, more preferably warm water. Most preferably the composition is a powder to be reconstituted in warm water.

Preferably the composition described herein is a nutritional composition.

### Scoring Method

In a preferred embodiment, the use of the combination of γ-aminobutyric acid and jujube in the manufacture of a composition for regulating the autonomic nervous system in a human subject further comprises a scoring method for regulating the autonomic nervous system in a human subject, comprising administering daily to said human subject a composition comprising
(i) γ-aminobutyric acid,
(ii) jujube, and
   wherein the method further comprises applying a wellbeing scoring system with the following steps:
a. obtaining user information from the human subject;
b. analyzing the obtained user information to obtain a wellbeing analysis;
c. based on the outcome of the wellbeing analysis:
   providing a wellbeing score to the human subject; and/or
   providing one or more suggestions for behavioral change to the human subject;
      wherein steps a. to c. are repeated at least daily.

In an alternative preferred embodiment, the use of the combination of γ-aminobutyric acid and jujube in the manufacture of a composition for treating and/or preventing one or more of stress, anxiety, depression, and memory loss in a human subject further comprises a scoring method for regulating the autonomic nervous system in a human subject, comprising administering daily to said human subject a composition comprising
(i) γ-aminobutyric acid,
(ii) jujube, and
   wherein the method further comprises applying a wellbeing scoring system with the following steps:
a. obtaining user information from the human subject;
b. analyzing the obtained user information to obtain a wellbeing analysis;
c. based on the outcome of the wellbeing analysis:
   providing a wellbeing score to the human subject; and/or
   providing one or more suggestions for behavioral change to the human subject;
wherein steps a. to c. are repeated at least daily.

A third aspect of the invention pertains to a scoring method for regulating the autonomic nervous system in a human subject, comprising administering daily to said human subject a composition comprising
(i) γ-aminobutyric acid,
(ii) jujube, and
   wherein the method further comprises applying a wellbeing scoring system with the following steps:
a. obtaining user information from the human subject;
b. analyzing the obtained user information to obtain a wellbeing analysis;
c. based on the outcome of the wellbeing analysis:
   providing a wellbeing score to the human subject; and/or
   providing one or more suggestions for behavioral change to the human subject;
      wherein steps a. to c. are repeated at least daily.

The inventors have found that the scoring method may enhance and/or maintain the physical and mental situation of the human subject and may therefore strengthen the positive effect of the claimed composition on regulating the autonomic nervous system and thereby strengthens the effect on treating and/or preventing one or more of hyperlipidaemia, hypertension, diabetes, obesity/overweight, stress, anxiety, depression, and memory loss in a human subject, preferably the effect on treating and/or preventing one or more of stress, anxiety, depression and memory loss in a human subject.

Preferably the wellbeing scoring system is a computer-implemented system and is preferably processed by a portable device.

The term “wellbeing score” as used herein refers to a score on a scale, e.g. of 1-10, wherein the wellbeing score is in an indication of how healthy the subject is overall. Preferably, the higher the score, the higher the wellbeing. The continuous adaptation of the wellbeing score in the scoring method provides feedback to the human subject whether the consumption of the composition and/or the changes in behaviour result in improved wellbeing. The wellbeing score preferably may be a recognition of user behaviour and may also be a psychological suggestion, allowing users to feel satisfied with their self-discipline and enter a virtuous cycle of physical and mental health improvement.

The term “user information” as used herein refers to information relating to the behaviour of the human subject and to information regarding the characteristics of the human subject.

Examples of information relating the behaviour of the human subject are one or more of dietary habits, sleeping habits, stool patterns, menstruation cycle/pregnancy/lactation, exercise routines, meditation habits, and the like.

Examples of information regarding the characteristics of the human subject are one or more of age, weight, length, gender, dietary restrictions, allergies, genetic information, family history, location information and the like.

Preferably, all embodiments with regards to the first and second aspect of the invention equally apply to the third aspect of the invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### Example 1

### Study Participants

200 study participants were selected in 8 cities in China. The study participants were aged 40 to 60 years old, with a ratio of over 50 years old to under 50 years old of 1:1. The ratio of male to female participants was 1:1. Over 50 years old female participants were perimenopausal or menopausal. Participants had not been diagnosed with the following issues: severe mental and physical health problems, severe sleep problems, drug or alcohol abuse, diabetes, hypertension, or cardiovascular diseases. Participants were not allergic to dairy nor lactose intolerant. Participants did not take any sleeping aid pills or supplements.

### Methodology

The study participants consumed daily a composition as described in Table 1 for 14 consecutive days. The composition was ingested within one hour before going to bed. The participants completed an online questionnaire on day 0 (before consumption) and in the morning on days 7 and 14. In the questionnaire the participants rated symptoms on a 5 point scale (1-5).

TABLE 1
Components and daily dosages of the composition of the invention

| Component | Daily dosage | |
|---|---|---|
| γ-aminobutyric acid | 100 | mg |
| Jujube seed powder | 500 | mg |
| Polyunsaturated fatty acids | | |
| Docosahexaenoic acid | 11 | mg |
| Eicosapentaenoic acid | 14 | mg |
| Vitamins | | |
| Folate | 136 μg DFE | |
| Vitamin B6 (α-TE) | 0.35 | mg |
| Vitamin B12 | 0.8 | μg |
| Vitamin C | 30 | mg |
| Vitamin E | 2.5 mg α-TE | |
| Protein | 5.5 | g |
| Fat | 6.75 | g |
| Carbohydrate | 10.25 | g |
| Energy | 123.8 | kcal |

### Results

Results are reported in Tables 2-4.

TABLE 2
Thermoregulation

| | Temperature | Feeling too hot | Feeling too cold |
|---|---|---|---|
| | related discomfort * | (mean times | (mean times |
| Time | (score 1-5 scale) | per week) | per week) |
| Day 0 | 1.8 | 0.8 | 0.6 |
| Day 7 | 1.4 | 0.7 | 0.2 |
| Day 14 | 1.3 | 0.5 | 0.1 |

* Symptoms related to temperature related discomfort are e.g. dry mouth, hot rash, excessive sweating, and goose bumps

TABLE 3
Respiratory

| | Respiratory | Can't breathe | Snoring loudly |
|---|---|---|---|
| | related discomfort * | comfortably | (mean times |
| Time | (score 1-5 scale) | (mean times per week) | per week) |
| Day 0 | 1.7 | 0.5 | 1.5 |
| Day 7 | 1.3 | 0.3 | 1.1 |
| Day 14 | 1.2 | 0.1 | 0.6 |

* Symptoms related to respiratory related discomfort are e.g. chest tightness, feeling of suffocation, and difficulty breathing

TABLE 4
Depressed mood, anxious mood, tension

| | Depressed mood | Anxious mood | Tension | Memory loss |
|---|---|---|---|---|
| | (score 1-5 | (score 1-5 | (score 1-5 | (score 1-5 |
| Time | scale) | scale) | scale) | scale) |
| Day 0 | 2.5 | 2.5 | 2.3 | 2.9 |
| Day 7 | 1.8 | 1.8 | 1.7 | 2.2 |
| Day 14 | 1.5 | 1.6 | 1.4 | 1.8 |

Taken together these results indicate an improved regulation of autonomic nervous system. In one aspect, the regulation of autonomic nervous system results improved thermoregulation. As shown in Table 2, improved thermoregulation is bidirectional, meaning that it encompasses both increasing and reducing body temperature. In another aspect, the improved regulation of autonomic nervous system results in improved breathing and a reduction of snoring. In yet another aspect, improvement of depressed mood, anxious mood, tension and memory loss are shown in Table 4.

1.-17. (canceled)

18. A method for regulating the autonomic nervous system in a human subject, the method comprising administering a composition comprising a combination of
(i) γ-aminobutyric acid,
(ii) jujube,
wherein the composition further comprises dairy protein and wherein the weight ratio of y-aminobutyric acid (mg) to protein (g) is in the range of 30:1-5:1.

19. The method according to claim 19, wherein the composition is a nutritional composition.

20. The method according to claim 19, wherein regulating the autonomic nervous system is improving thermoregulation.

21. The method according to claim 20, wherein improving thermoregulation is maintaining body temperature around physiological body temperature.

22. The method according to claim 20, wherein the human subject is one or more of a human subject suffering of hormonal imbalance, a human subject undergoing hormonal treatments, a human subject who is underweight, a human subject who is overweight, or an aging human subject.

23. The method according to claim 5, wherein the human subject suffering of hormonal imbalance is selected from a woman in perimenopausal period or a woman in menopausal period.

24. The method according to claim 18, wherein regulating the autonomic nervous system is improving breathing and/or treating snoring.

25. The method according to claim 18, wherein jujube is provided as an extract derived from jujube fruit, jujube leaves, jujube seed, or combinations thereof.

26. The method according to claim 18, wherein the weight ratio of γ-aminobutyric acid (mg) to jujube (mg) is in the range of 2:1-1:10.

27. The method according to claim 18, wherein the amount of γ-aminobutyric is 100-1000 mg per 100 g of dry weight of the composition.

28. The method according to claim 18, wherein the amount of jujube is 0.1-6 g per 100 g of dry weight of the composition.

29. The method according to claim 18, wherein the composition comprises one or more of docosahexaenoic acid (DHA), eicosapentaenoic acid (EPA), and docosapentaenoic acid (DPA).

30. The method according to claim 29, wherein the weight ratio of DHA (mg) to EPA (mg) is in the range of 5:1-1:4.

31. The method according to claim 18, wherein the composition comprises one or more vitamins selected from folate, vitamin B6, vitamin B12, vitamin C, and vitamin E.

32. The method according to claim 18, wherein the use further comprises a scoring method for regulating the autonomic nervous system in a human subject, comprising administering daily to said human subject a composition comprising
(i) γ-aminobutyric acid,
(ii) jujube, and
wherein the method further comprises applying a wellbeing scoring system with the following steps:
a. obtaining user information from the human subject;
b. analyzing the obtained user information to obtain a wellbeing analysis;
c. based on the outcome of the wellbeing analysis:
providing a wellbeing score to the human subject; and/or
providing one or more suggestions for behavioral change to the human subject;
wherein steps a. to c. are repeated at least daily.

33. A method for treating and/or preventing one or more of stress, anxiety, depression, and memory loss in a human subject, the method comprising administering a composition comprising a combination of
(i) γ-aminobutyric acid,
(ii) jujube.

34. The method according to claim 33, wherein the use further comprises a scoring method for regulating the autonomic nervous system in a human subject, comprising administering daily to said human subject a composition comprising
(i) γ-aminobutyric acid,
(ii) jujube, and
wherein the method further comprises applying a wellbeing scoring system with the following steps:
a. obtaining user information from the human subject;
b. analyzing the obtained user information to obtain a wellbeing analysis;
c. based on the outcome of the wellbeing analysis:
providing a wellbeing score to the human subject; and/or
providing one or more suggestions for behavioral change to the human subject;
wherein steps a. to c. are repeated at least daily.
